(19) United States
(12) Patent Application Publication
(10) Pub. No.: US 2023/0077429 A1
(43) Pub. Date: Mar. 16, 2023

(54) CELL-CULTURE BIOREACTOR

(71) Applicant: Orgenesis Inc., Germantown, MD (US)

(72) Inventors: VERED CAPLAN, COPPET (CH); MICHAEL FAINSHTEIN, MAPLE (CA)

(73) Assignee: Orgenesis Inc., Germantown, MD (US)

(21) Appl. No.: 17/904,549

(22) PCT Filed: Feb. 18, 2021
(86) PCT No.: PCT/US21/18465

§ 371 Date: Aug. 18, 2022

Related U.S. Application Data
(60) Provisional application No. 62/978,057, filed on Feb. 18, 2020.

(51) Int. Cl. C12M 1/00; C12M 1/06; C12M 1/02; C12M 1/34

(52) U.S. Cl. CPC C12M 29/08; C12M 27/06; C12M 41/18; C12M 41/42; C12M 41/34

(57) ABSTRACT

A cell-culture bioreactor operative to provide real-time reactor management in the fields of stir control, sparge control, and heat management responsively to wireless sensor feedback to optimize operating conditions to maximize cell-culture yield; and a bioreactor comprising: a vessel, having an open top; a headplate, configured to seal the vessel's open top; and a stirring device, comprising at least two independent stirring elements; wherein each of the stirring elements is configured to independently stir media accommodated within the vessel.

### FIELD OF THE INVENTION

The presently disclosed subject matter relates to vessels for biological liquids such as cell-culture bioreactors, and in particular to stirring mechanisms therefor.

### BACKGROUND OF THE INVENTION

Cell culture generally involves the removal of cells from an animal or plant and culturing the cells in a favorable artificial environment. Culture conditions vary widely for each cell type, but the artificial environment in which the cells are cultured typically involves the use of a suitable vessel containing: a substrate or medium that supplies the essential nutrients (such as amino acids, carbohydrates, vitamins, minerals); growth factors; hormones; gases (such as O₂, CO₂); and a regulated physico-chemical environment (including regulation of pH, osmotic pressure, temperature, etc.). Some cells must be cultured while attached to a solid or semi-solid substrate (adherent or monolayer culture), while others can be grown floating in the culture medium (suspension culture). The expanded cell cultures can then be used in further bioprocessing in the production of therapeutic products.

A bioreactor can be employed in a bioprocess to optimize cell culture, with many factors influencing this optimization, including dissolved oxygen (DO) and carbon dioxide (CO₂) levels. Oxygen is crucial for the cellular processes of respiration and cell division and CO₂ is a waste byproduct of these processes. Both DO and CO₂ can impact the culture media pH and the product quality and thus these levels need to be controlled. Within a bioreactor, agitation (through the use of impellers) and aeration (e.g., via sparging) are used to control DO levels at desired values. However, agitation and sparging also cause shear stress on the cells, which may negatively affect cell culture yield.

Accordingly, there remains a need in the art for cell culture bioreactors with improved designs that overcome these and other shortcomings of those known in the art.

### SUMMARY

According to some embodiments of the presently disclosed subject matter, a new bioreactor is provided comprising:
a vessel, defining a cavity therewithin; and
a stirring device, comprising at least two stirring elements; the stirring device being configured to operate each of the stirring elements independently of the other to stir media accommodated within the cavity, said stirring elements being configured to rotate about a common stirring axis.

According to some embodiments, each stirring element comprises a cylindrical stirring shaft attached at its distal end to an impeller located within the cavity, and at its proximal end to a motor, located exterior to the vessel; each motor is configured to independently rotate and/or translate its associated stirring shaft around and/or along the stirring axis, and accordingly its associated stirring impeller.

According to some embodiments, at least one stirring shaft comprises a hollow cylindrical shape, configured to at least partially accommodate, along its longitudinal axis, at least one other stirring shaft, in a telescopic cylinder configuration, configured to enable the rotation and translation of the at least one other stirring shaft therewithin, and such that their associated impellers reside one next to the other, along the stirring axis.

According to some embodiments, the bioreactor is configured to be accommodated on a table construction; the table construction comprises at least one motor shaft per each motor, such that each motor is configured to be applied on and move along its at least one associated motor shaft, and optionally at least one mutual shaft.

According to some embodiments, the bioreactor further comprising a sleeve tube configured to cover and seal a section of the stirring shafts, the section accommodated within the vessel, such that the stirring shafts are isolated from the vessel's accommodated media, wherein:
the sleeve tube is configured to enable the rotation and translation of the stirring shafts therewithin; and
the impellers are configured to be threaded around the exterior of the sleeve tube; the impellers attachment with the distal end of their associated stirring shafts is via magnetic communication, through the sleeve tube, such that the impellers are enabled to rotate and translate together with their associated stirring shafts.

According to some embodiments, the sleeve tube comprises a ring bracket at its exterior distal end, configured to prevent the impellers release from the sleeve tube.

According to some embodiments, the sleeve tube is disposable.

According to some embodiments, the sleeve tube is at least partially transparent.

According to some embodiments, the impellers are disposable.

According to some embodiments, the stirring shafts are configured to pass through the headplate, such that media accommodated in the vessel remains sealed therewithin.

According to some embodiments, at least one stirring shaft is attached to its associated impeller via a magnetic coupling.

According to some embodiments, at least one stirring shaft is attached to its associated motor via a magnetic coupling and/or a belt coupling.

According to some embodiments, the stirring device is configured to operate the stirring elements to move along the stirring axis independently of one another or to rotate at different rotational velocities than each other.

According to some embodiments, the stirring device is configured to operate the stirring elements to move along the stirring axis independently of one another and to rotate at different rotational velocities than each other.

According to some embodiments, the bioreactor further comprising a sparger.

According to some embodiments, the bioreactor further comprising sensing elements attached to the vessel's interior surface and their associated readers attached to the vessels exterior surface.

According to some embodiments, the vessel comprising plurality of ports configured to enable at least one or more selected from the group including: harvesting, washing, sensing, sampling, media refreshing, gas venting, and seeding.

According to some embodiments, the bioreactor further comprising at least one controller configured to enable automatic maintenance of an accommodated media.

According to some embodiments, the vessel being at least partially transparent.

According to some embodiments, the vessel being disposable.

According to some embodiments of the presently disclosed subject matter, a new stirring device is provided, comprising at least two stirring elements; the stirring device being configured to operate each of the stirring elements independently of the other to stir fluid accommodated within a vessel, all the stirring elements being configured to rotate around a common stirring axis.

According to some embodiments, each stirring element comprises a cylindrical stirring shaft attached at its distal end to an impeller, and at its proximal end to a motor; each motor is configured to independently rotate and/or translate its associated stirring shaft around and/or along the stirring axis, and accordingly its associated impeller.

According to some embodiments, at least one stirring shaft is attached to its associated impeller via a magnetic coupling.

According to some embodiments, at least one stirring shaft is attached to its associated motor via a magnetic coupling and/or a belt coupling.

According to some embodiments, at least one stirring shaft comprises a hollow cylindrical shape, configured to at least partially accommodate along its longitudinal axis at least one other stirring shaft, in a telescopic cylinder configuration, configured to enable the rotation and translation of the at least one other stirring shaft therewithin, and such that their associated impellers reside one next to the other, along the stirring axis.

According to some embodiments, the device is configured to be accommodated on a table construction; the table construction comprises at least one motor shaft per each motor, such that each motor is configured to be applied on and move along its at least one associated motor shaft, and optionally at least one mutual shaft.

According to some embodiments, the device further comprising, a sleeve tube configured to cover and seal a section of the stirring shafts, wherein:
the sleeve tube is configured to enable the rotation and translation of the stirring shafts therewithin; and
the impellers are configured to be threaded around the exterior of the sleeve tube; the impellers attachment with the distal end of their associated stirring shafts is via magnetic communication, through the sleeve tube, such that the impellers are enabled to rotate and translate together with their associated stirring shafts.

According to some embodiments, the sleeve tube comprises a ring bracket at its exterior end, configured to prevent the impellers release from the sleeve tube.

According to some embodiments, the stirring device is configured to operate the stirring elements to move along the stirring axis independently of one another or to rotate at different rotational velocities than each other.

According to some embodiments, the stirring device is configured to operate the stirring elements to move along the stirring axis independently of one another and to rotate at different rotational velocities than each other.

According to some embodiments of the presently disclosed subject matter, a new bioreactor is provided comprising:
a vessel, defining a cavity therewithin; and
a stirring device, comprising at least one stirring element, the stirring device being configured to operate said stirring element to stir media accommodated within the cavity, said stirring element being configured to rotate about and/or translate along a stirring axis; wherein, the stirring element comprises a cylindrical stirring shaft attached at its distal section to at least one impeller located within the cavity, and at its proximal section to a motor, located exterior to the vessel; the motor is configured to rotate and/or translate the stirring shaft around and/or along the stirring axis, and accordingly its stirring impeller/s;
a sleeve tube configured to cover and seal a section of the stirring shaft, the section accommodated within the vessel, such that the stirring shafts is isolated from the vessel's accommodated media, wherein:
   the sleeve tube is configured to enable the rotation and translation of the stirring shaft therewithin; and
   the impeller/s is/are configured to be threaded around the exterior of the sleeve tube; the impeller/s attachment with the distal section of the stirring shaft is via magnetic communication, through the sleeve tube, such that the impeller/s is/are enabled to rotate and translate together with the stirring shaft.

According to some embodiments, the sleeve tube comprises a ring bracket at its exterior end, configured to prevent the impeller/s release from the sleeve tube.

According to some embodiments of the presently disclosed subject matter, a new bioreactor is provided, comprising:
a vessel having an open top and a vessel wall;
heating and/or cooling elements embedded in the vessel walls;
one or more control elements associated with the heating and/or cooling elements located outside the vessel;
a reactor headplate configured to close the open top of the vessel to the atmosphere;
at least one stirrer shaft in the vessel magnetically coupled to a motor mounted on the headplate outside the vessel; and
a sparger, comprising:
   a tube assembly inside the vessel, having a gas outlet;
   a source of sparging gas outside the vessel in fluid communication with the tube assembly; and
   a sparger driver motor outside the vessel configured to control insertion of gas into the tube assembly.

According to some embodiments, the vessel walls comprise: glass, polymer, composite material, or a combination thereof.

According to some embodiments, the vessel walls comprise a layered construction.

According to some embodiments of the presently disclosed subject matter, a new bioreactor is provided, comprising:
a vessel having an open top and a vessel wall;
a reactor headplate closing the open top of the vessel to the atmosphere;
at least one magnetic stirrer shaft in the vessel, magnetically coupled to at least one motor mounted on the headplate outside the vessel; the magnetic stirrer shaft comprising a plurality of impellers, at least one impeller of said plurality of impellers having an adjustable vertical position on the stirrer shaft;
a sparger, comprising:
   a tube assembly inside the vessel, having an outlet inside the tube;
   a source of sparging gas outside the vessel;
   a conduit, sealed to the atmosphere, opening to the source of sparging gas and to the tube assembly; and
   a sparger driver motor outside the vessel configured to control gas insertion into the tube assembly.

According to some embodiments, the vertical position on the stirrer shaft is controlled from outside the vessel.

According to some embodiments of the presently disclosed subject matter, a new bioreactor is provided, comprising:
a vessel having an open top and a vessel wall;
a reactor headplate closing the open top of the vessel to the atmosphere;
heating and/or cooling elements embedded in the vessel walls;
controls associated with the heating and/or cooling elements located outside the vessel;
a stirrer shaft in the vessel magnetically coupled to a stirrer motor mounted on the headplate outside the vessel;
controls associated with the stirrer motor;
a sparger, comprising a tube assembly inside the vessel, in fluid communication with a source of sparging gas and having an outlet into the vessel; and
a sensor array in fluid communication with the vessel contents and in operative communication with the heating and/or cooling element controls and with the stirrer motor controls.

According to some embodiments, the bioreactor further comprising controls associated with the sparger, wherein the sensor array is in operative communication with the outlet in the sparger to control the rate of the outlet of sparging gas.

According to some embodiments, the sensor array includes at least two different sensors selected from: temperature sensor, pH sensor, dissolved oxygen sensor, glucose sensor, lactate sensor, and cell count sensor, and signals from said sensor array actuate changes in heating and or cooling, impeller speed or direction, and rate and size of bubbles generated in the vessel.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIGS. 1A and 1B are schematic views of a bioreactor, according to various embodiments of the presently disclosed subject matter;

FIGS. 1C through 1E are schematic exploded views of a bioreactor and a stirring device, according to various embodiments of the presently disclosed subject matter;

FIG. 1F is a schematic cut view of a bioreactor, according to some embodiments of the presently disclosed subject matter;

FIG. 2 is a schematic side view of a lab on chip coupled with a hydro-focusing system, according to some embodiments of the presently disclosed subject matter;

FIG. 3 is a block diagram of the bioreactor system overview depicting controller hardware, software, and reactor hardware, according to some embodiments of the presently disclosed subject matter; and

FIG. 4 is a process flow diagram for embodiments employing sensor responsive run-time, parameter optimization, according to some embodiments of the presently disclosed subject matter.

It will be appreciated that for the sake of clarity, elements shown in the figures may not be drawn to scale and reference numerals may be repeated in different figures to indicate corresponding or analogous elements.

### DETAILED DESCRIPTION

The present subject matter may be understood more readily by reference to the following detailed description which forms a part of this disclosure. It is to be understood that the invention is not limited to the specific products, methods, conditions or parameters described and/or shown herein, and that the terminology used herein is for the purpose of describing particular embodiments by way of example only and is not intended to be limiting of the claimed invention. Further, unless otherwise required by context, singular terms shall include pluralities and plural terms shall include the singular.

Embodiments of the presently disclosed subject matter are directed, inter alia, to a bioreactor providing real-time control in a variety of areas, such as stir control, sparging, and heat management, for example. This added real-time control enables optimization of operation parameters during run time to maximize cell-culture yield.

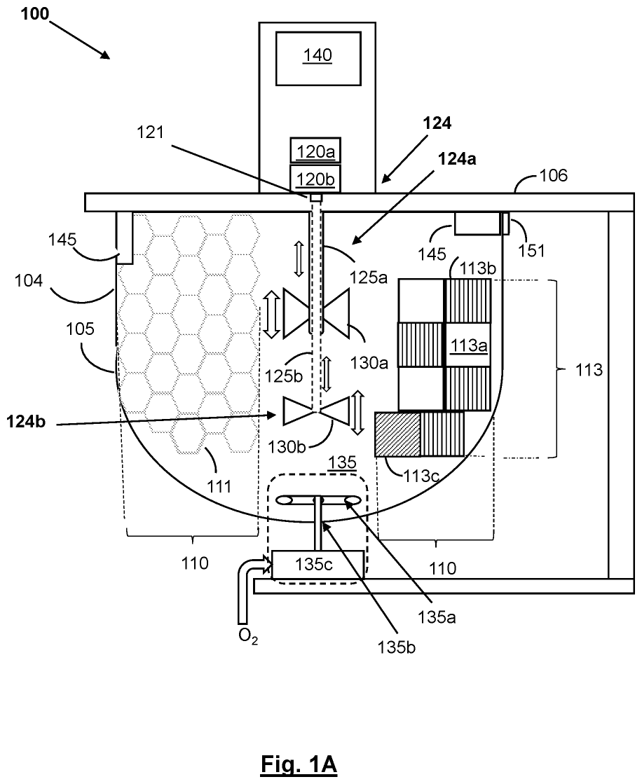

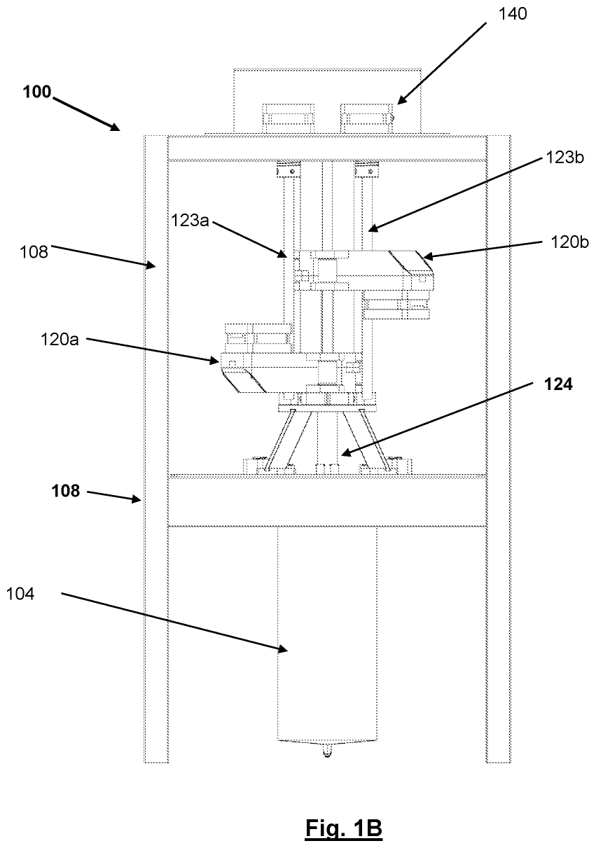

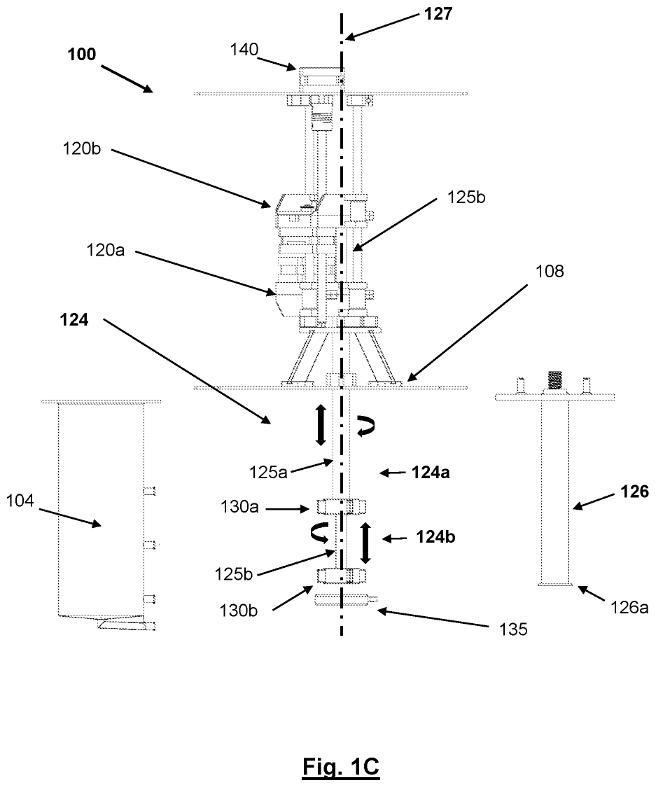

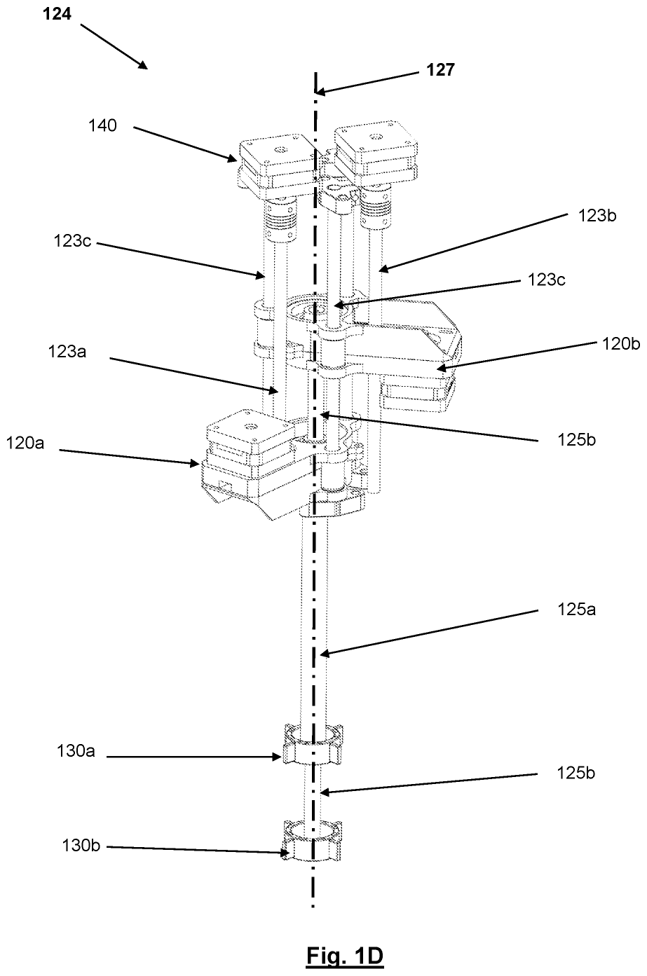

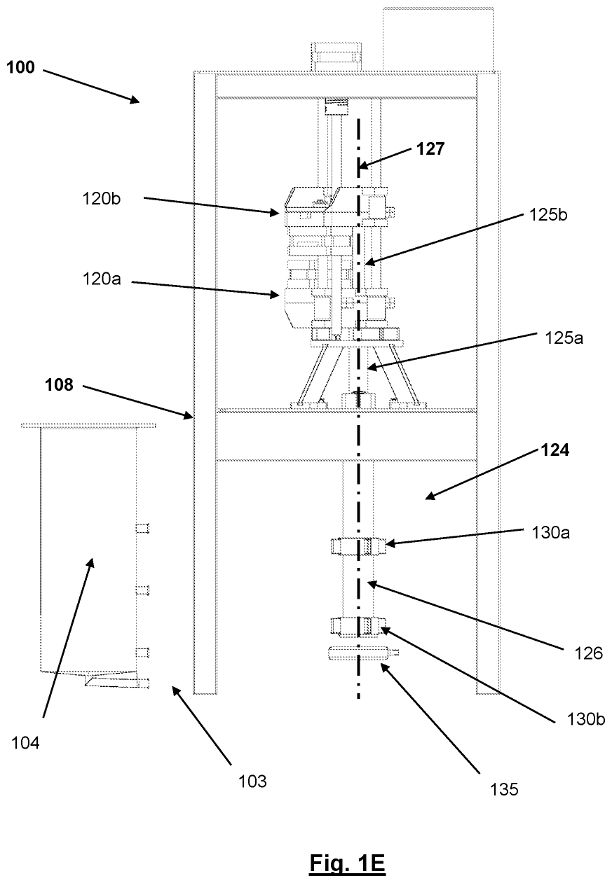

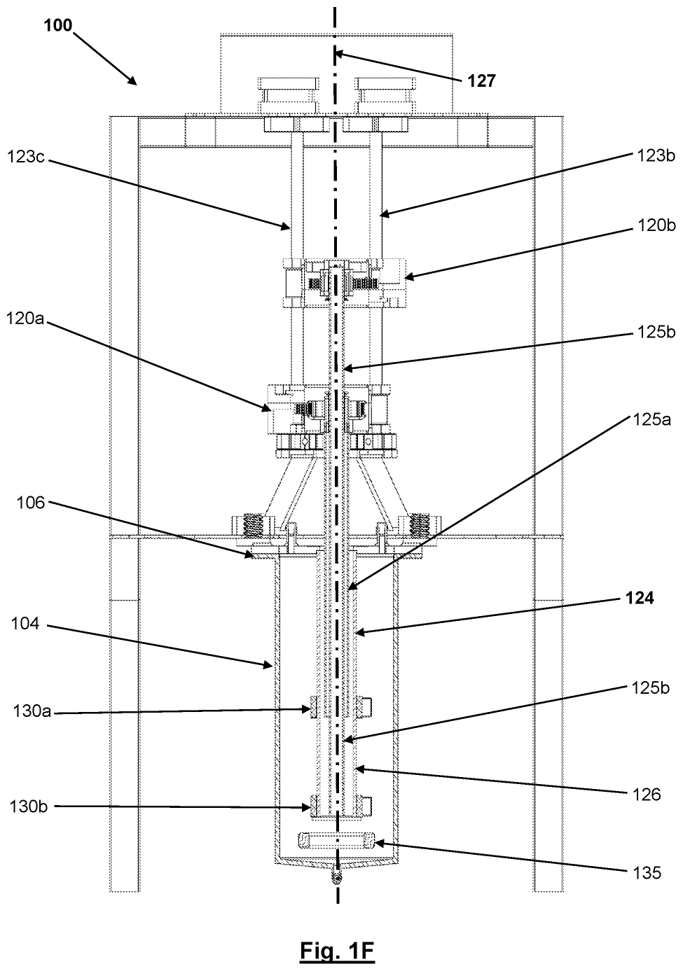

Turning now to the figures, FIG. 1A is a schematic, cross-sectional side view of a cell-culture bioreactor 100, according to some embodiments of the presently disclosed subject matter, having a vessel 104 with housing wall 105 defining therewithin a cavity, an orifice-free reactor cover or headplate 106, a heat management system 110 including a heating element grid 111 and a Peltier thermocouple array 113, a variable-speed, reversible motor/s 120a, 120b in magnetic communication with impeller shafts (i.e., stirring shafts) 125a, 125b, a primary impeller 130a and a secondary impeller 130b, each slidably mountable to impeller shafts 125a and 125b, respectively, a sparger assembly 135, a sensor array 145, at least one controller 140 in communication with motors 120a and 120b, and a sparger assembly 135.

According to some embodiments, the housing wall 105 constructed from glass, in other embodiments, a polymeric material is used, alone or in combination with glass. For example, in some embodiments, the vessel is a polymeric shell with an inner glass lining. In other embodiments metallic materials, ceramics or composites are used.

### Heat Management

As shown, bioreactor 100 is fitted with a heat management system 110 of heating units, advantageously providing real-time and area-specific heat management, configured to advantageously enable heat management during a run time, to maximize cell-yield.

In a certain embodiment, the heat management system 110 is formed from heating units, either from wire heating elements, or from Peltier thermocouples, or both.

As shown, the housing is embedded with heating grid 111, formed from a plurality of heating elements and fitted with an array of Peltier thermocouples 113 in thermal communication with housing wall 105. As shown, heating grid 111 is formed from one or more heating cells to advantageously provide an independent and an area-specific heating. In a certain embodiment, each heating cell is individually controlled, whereas in another embodiment a plurality of heating cells is controlled collectively. (Some segments of heating grid 111 are not depicted for the sake of clarity.)

In a certain embodiment, the heating cells have a pentagonal geometry, whereas in another embodiment the cells have a square geometry. It should be appreciated that various in embodiments, other polygonal heating cell geometries are employed.

As noted above, heat management system 110 includes an array of Peltier thermocouples operative to either heat or cool the housing wall 105 in accordance with the voltage applied. As shown, thermocouples 113a and 113b are driven by reversible voltage polarity and therefore either heat or cool the housing wall 105 and the vessel contents. As shown, thermocouple 113a can be operative to heat the housing wall 105 and thermocouple 113b can be operative to cool the housing wall 105 and to discharge heat to a heat sink in accordance with the set voltage polarity driving them. According to some embodiments, thermocouple 113c can be driven by reversible voltage polarity and therefore is operative to both cool and to heat in accordance with the voltage polarity applied. According to some embodiments, the voltage polarity applied to thermocouples 113a-113c is set by controller 140 as is the voltage magnitude applied to both heating grid and thermocouple array components. According to some embodiments, controller 140 is configured to manage heat responsively to a sensor feedback, or in accordance with preset guidelines, or a combination of sensor feedback and preset guidelines. According to some embodiments, heat management system 110 is manageable in terms of temperature, heated/cooled area of the housing wall, timing, and time period. In another embodiment, heat management system 110 is implemented only with wire heating elements, whereas in another embodiment the heat management system 110 is implemented only with Peltier thermocouples, as noted above. Alternatively, or in combination, heating and cooling may be provided with a conventional jacketed reactor system comprising a circulating heat transfer fluid circulating in a jacket around at least a portion of housing wall 105.

### Stirring

According to some embodiments, for example as illustrated in FIG. 1A, bioreactor 100 is fitted with one or more impeller shafts 125a and 125b coupled to respective drive motors 120a and 120b; according to some embodiments, through magnetic coupling 121. According to some embodiments, reactor-cover or headplate 106 is made of a non-magnetic material, to facilitate the magnetic coupling that advantageously eliminates the danger of culture contamination from a cover orifice traversed by an impeller shaft. Examples of various embodiments of magnetic coupling 121 to motors 120a and 120b include integrally magnetized stirrer shafts 125a and 125b and/or magnetic coupling elements fastened to stirrer shafts 125a and/or 125b and motor shafts of motors 120a and 120b.

In some embodiments, a single impeller shaft 125a is employed, and the primary and secondary impellers, 130a and 130b are slidably mounted to shaft 125a to configured to enable selection of impeller placement to generate desired stir characteristics. Impellers, 130a and 130b are secured at the desired shaft height by way of connection configurations like resiliently biased clips, tabs or Allen bolt and other releasable connection configurations known to those skilled in the art. In some embodiments, height of one or more of the impellers is controlled remotely without removing cover 106.

In some embodiments a plurality of impeller shafts is employed, and the shafts are concentrically disposed. For example, shaft 125a is implemented as a tube shaft driving impeller 130a, while inner impeller shaft 125b drives impeller 130b, so as to provide separate impeller-specific speed, direction, and timing control. In a certain embodiment inner impeller shaft 125b is vertically conveyable within tube impeller shaft 125a through vertical conveyance of a magnetically coupled second motor 120a.

According to some embodiments, mixing can be influenced by various factors, including media viscosity, total media volume, bioreactor dimensions, agitation and stirring speeds, and impeller design and shape. In some embodiments of the bioreactors provided herein, impellers are configured to employ unique impeller configurations that impart improved functionality in these processes.

According to some embodiments, impellers 130a or 130b are either both implemented as radial impellers in a certain embodiment, or as linear impellers in another embodiment, or as a linear and a radial embodiment in another embodiment. According to some embodiments, axial impellers are best for mixing applications that require stratification or solid suspension. According to some embodiments, axial impellers are set up to create effective top to bottom motion in the tank.

According to some embodiments, radial impellers employ 4-6 blades that drive perpendicularly to the impeller. The resulting radial flow pattern moves contents to the sides of the bioreactor and further drives the contents vertically to the cover plate 106 or to the bottom of reactor's housing 105. In a certain embodiment, two or more impellers are employed, whereas in another embodiment only one impeller is employed.

According to some embodiments, rotational velocities and directions are defined by motors 120a, 120b and controlled by at least one controller 140. In a certain embodiment, rotational velocity, location, and direction are defined by sensor data, supplied by one or more sensors in sensor array 145, in accordance with configuration parameters stored in controller/s 140.

According to some embodiments of the presently disclosed subject matter, and as illustrated in FIGS. 1B through 1F, a bioreactor 100 is provided. The bioreactor 100 comprises:
a vessel 104 defining therewithin a cavity, the vessel having an open top and comprising a headplate 106 configured to seal the open top; and
a stirring device 124, comprising at least two independent stirring elements 124a, 124b; wherein each of the stirring elements is configured to independently stir media accommodated within the cavity defined by the vessel.

It will be appreciated that the while the bioreactor 100 is described herein and illustrated in the accompanying figures as comprising a vessel 104 and a headplate 106, this is by way of non-limiting example only. In practice, the bioreactor 100 may comprise a sealed vessel which is closed on the top, and for which access to the cavity defined therein is provided via other means, for example through ports, a side hatch, etc., without departing from the scope of the presently disclosed subject matter, mutatis mutandis. Similarly, although the stirring device 124 is described below as passing through the headplate 106, this is not to be construed as limiting, and it may pass into the cavity of the vessel 104 via any suitable portion thereof, and at any suitable orientation, e.g., a non-vertical orientation.

According to some embodiments, all of the stirring elements 124a, 124b are configured to rotationally stir the media around the same stirring axis 127 (illustrated in FIGS. 1C through 1E).

According to some embodiments, the stirring device is configured to operate the stirring elements to move along the stirring axis 127 independently of one another and/or to rotate at different rotational velocities around the stirring axis 127 (including direction, wherein, e.g., a rotational velocity in a clockwise direction may be considered having a positive rotational velocity, and a rotational velocity in a counterclockwise direction may be considered having a negative rotational velocity) than each other, including varying their rotational velocities independently of each other.

According to some embodiments, each stirring element 124a, 124b comprises a stirring cylindrical shaft 125a, 125b attached at or near its distal end (e.g., bottom end) to an associated impeller 130a, 130b, which located within the vessel, and attached at or near its proximal end (e.g., top end) to a motor 120a, 120b, located out of the vessel (e.g., above the headplate 106); each motor 120a, 120b is configured to independently rotate and/or translate its associated stirring shaft 125a, 125b around and/or along the stirring axis 127, and accordingly its associated impeller 130a, 130b. According to some embodiments, the motor can be a stepper motor (for example an electric motor that divides a full rotation into number of equal steps) and/or a linear actuator that creates motion in a straight line.

According to some embodiments, for example as illustrated in FIG. 1F, the stirring shafts 125a, 125b are configured to pass through the headplate 106, such that media accommodated in the vessel 104 remains sealed therewithin.

According to some embodiments, at least one stirring shaft is attached to its associated impeller via a magnetic coupling. According to some embodiments, both the distal end of the stirring shaft and its associated impeller comprise magnetic material/s adapted for said magnetic coupling.

According to some embodiments, at least one stirring shaft is attached to its associated motor via a magnetic coupling and/or a belt coupling. According to some embodiment, both the proximal end of the stirring shaft and its associated motor comprise magnetic material/s adapted for said magnetic coupling.

According to some embodiments, at least one stirring shaft comprises a hollow cylindrical shape, configured to at least partially accommodate at least one other stirring shaft along its longitudinal axis, for example in a telescopic cylinder configuration (i.e., shafts are concentric at stirring axis), configured to enable the rotation and translation of the other stirring shaft/s therewithin, and such that their associated impellers reside one next to the other, along the stirring axis 127. In the example described with reference to and illustrated in FIGS. 1B through 1F, stirring shaft 125a comprises a hollow cylindrical shape configured to accommodate another stirring shaft 125b, in a telescopic cylinder configuration. The cylindrical telescopic configuration enables the rotation and translation of the other stirring shaft/s 125b therewithin, and such that their associated impellers 130a, 130b reside one next/above to the other, along the stirring axis 127.

According to some embodiments, the impeller's stirring wings can comprise a variety of shapes and variety of orientations, respective to the stirring axis.

According to some embodiments, the bioreactor is configured to be accommodated on a table construction 108, for example as illustrated in FIGS. 1B and 1E. The table construction comprises at least one motor shaft 123a, 123b per each motor 120a, 120b, such that each motor is configured to be applied on, and move along, its at least one associated motor shaft 123a, 123b, and optionally at least one mutual shaft 123c; the motor shafts 123a, 123b and the optional mutual shaft 123c are illustrated, e.g., in FIG. 1D.

According to some embodiments, and as illustrated in FIGS. 1C and 1E, the bioreactor further comprises at least one sleeve tube 126, configured to cover and seal a section of the stirring shafts 125a, 125b, the section accommodated within the vessel 104, such that the stirring shafts are isolated from the vessel's accommodated media. According to such embodiments:
the sleeve tube 126 is configured to enable the rotation—and translation—of the stirring shafts 125a, 125b (around and along the stirring axis 127) therewithin; and
the stirring shafts' associated impellers 130a, 130b are configured to be threaded around the exterior of the sleeve tube; wherein the impellers' attachment with the distal end (e.g., bottom end) of their associated stirring shafts 125a, 125b is via a magnetic communication, through the sleeve tube, such that the impellers are enabled to rotate—and translate—together with their associated stirring shafts.

According to some embodiments, for example as illustrated in FIG. 1C, the sleeve tube comprises a ring bracket 126a, at or near its exterior distal end (bottom end), configured to prevent the impellers release from the sleeve tube (e.g., configured to prevent the impellers falling from the bottom of the sleeve tube).

According to some embodiments, the sleeve tube is disposable. According to some embodiments, the sleeve tube is at least partially transparent. According to some embodiments, the impellers are disposable.

According to some embodiments, the bioreactor further comprises a sparger 135 configured to insert gas (e.g., oxygen) into the vessel 104. According to some embodiments, the bioreactor further comprises sensing elements (not shown) attached to the vessel's interior surface and their associated readers attached to the vessels exterior surface (not shown). According to some embodiments, the vessel 104 comprises plurality of ports 103, configured to enable at least one of: harvesting, washing, sensing, sampling, media refreshing, gas out letting, seeding and any combination thereof. According to some embodiments, the bioreactor further comprises at least one controller 140 configured to enable automatic maintenance of an accommodated media. According to some embodiments, the vessel 104 is at least partially transparent. According to some embodiments, the vessel 104 is disposable.

According to some embodiments of the presently disclosed subject matter, and as illustrated for example in FIGS. 1C through 1F, a stirring device is provided, configured to stir fluid accommodated within a vessel. The stirring device 124 comprising at least two independent stirring elements 124a, 124b, according to any one of the above-mentioned embodiments, and wherein each of the stirring elements is configured to independently stir fluid accommodated within the vessel, and wherein all the stirring elements 124a, 124b are configured to rotationally stir the fluid around the same stirring axis 127.

According to some embodiments of the presently disclosed subject matter, a bioreactor is provided comprising:
a vessel, defining a cavity therewithin; and
a stirring device, comprising at least one stirring element, the stirring device being configured to operate said stirring element to stir media accommodated within the cavity, said stirring element being configured to rotate about and/or translate along a stirring axis; wherein, the stirring element comprises a cylindrical stirring shaft attached at its distal section to at least one impeller located within the cavity, and at its proximal section to a motor, located exterior to the vessel; the motor is configured to rotate and/or translate the stirring shaft around and/or along the stirring axis, and accordingly its stirring impeller/s;
a sleeve tube configured to cover and seal a section of the stirring shaft, the section accommodated within the vessel, such that the stirring shafts is isolated from the vessel's accommodated media, wherein:
the sleeve tube is configured to enable the rotation and translation of the stirring shaft therewithin; and
the impeller/s is/are configured to be threaded around the exterior of the sleeve tube; the impeller/s attachment with the distal section of the stirring shaft is via magnetic communication, through the sleeve tube, such that the impeller/s is/are enabled to rotate and translate together with the stirring shaft.

According to some embodiments, the sleeve tube comprises a ring bracket at its exterior end, configured to prevent the impeller/s release from the sleeve tube.

### Sensing

According to some embodiments, bioreactor 100 is fitted with a variety of sensors, in sensor array 145 operative to detect a wide variety of parameters. Sensors may include, inter alia, temperature sensor, pH sensor, dissolved oxygen sensor, glucose sensor, lactate sensor, cell count sensor.

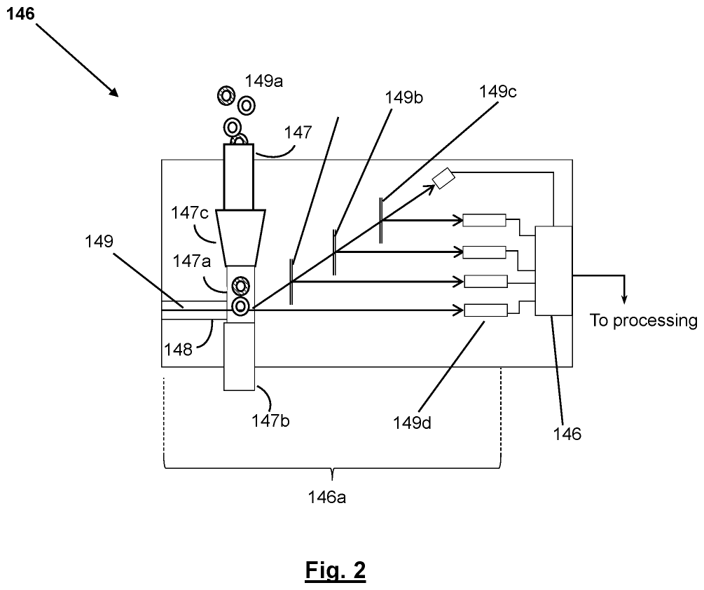

According to some embodiments, for example as illustrated in FIG. 2, sensor array 145 includes one or more remotely controlled integrated lab on chips (LOC) 146 coupled with a hydro-focusing system 146a that together is operative to perform cytometry measurements. The cytometric functionality advantageously enables counting of the desirable cells and even differentiation between desirable cells and undesirable cells. The LOC 146 is coupled with a hydro-focusing system 146a having one or more inlets 147, one or more outlet ports 147b, a flow cell 147a through which laser light 149 is directed from a laser releasable attached to a laser port 151 on chamber's wall 105 (FIG. 1A). Laser light 149 passing through cell passing through flow cell 147a both passes through the cells and is also scattered. The scattered light is directed through a plurality of wave guides disclosed at small angles of about 1° to 10° and also 90° relative to the direction of the flow stream through flow cell 147a. Laser beam 149 is further spectrally divided by different frequency-specific filters 149a-149c to cause fluorescence intensity over several fluorescence channels. Each light beam is then captured by its respective light detector 149d coupled to each wave guide, according to an embodiment. Detector output is then directed to LOC 146 for autofluorescence analysis thereby providing insight into cell identity and/or cell quantity as is known to those skilled in the art. LOC output is sent to processor/controller 140 where a suitable response is actuated responsively and, additionally or alternatively, to a user interface to provide feedback to relevant parties.

Hydro-focusing system 146a is operative to allow passage of cells individually in flow cell 147a through hydrodynamic focusing of the stream in jet 146c. It should be appreciated that in a certain embodiment fluoresce is not enhanced through the addition of fluorescence agents to prevent health complications when the cells are returned to the patient after processing. In another embodiment, patient compatible fluorescent dyes are introduced into the test sample.

The scattering intensity depends on the morphology of the cell (size, shape, internal structure), on the orientation of the cell in the flow relative to the direction of the incident radiation, and on the state of polarization of the incident radiation.

The fluorescence intensity is formed due to two contributions: specific fluorescence and autofluorescence. Specific fluorescence is associated with the emission of fluorochrome molecules that are specifically associated with certain cellular components (receptors, intracellular proteins, DNA, etc.). Autofluorescence is associated with the emission of the cell's own molecules (proteins, nucleic acids, etc.).

The intensity of specific fluorescence depends on the number of fluorochrome molecules in the cell, the wavelength of the exciting radiation, the absorption cross section (extinction) of the fluorochrome at this wavelength, the quantum yield of fluorochrome, the numerical aperture of the optical fluorescence collection system, and the spectral range of the optical filtering and detection system. The intensity of autofluorescence likewise depends on the optical properties of the intrinsic molecules of the cell. In practice, the preparation of cells before measurement is carried out in such a way that the contribution of specific fluorescence is several orders of magnitude greater than the contribution of autofluorescence.

In a certain embodiment, one or more of sensors 145 are in wireless communication with controller/s 140, such communication protocols may include, without limitation, WiFi, Bluetooth, or other suitable protocol for transmitting a signal. In another embodiment, the sensors are hard wired to controller/s 140, whereas in another embodiment some of the sensors are wirelessly connected to controller/s 140 and other sensors are hard wired with controller/s 140. In a certain embodiment, one or more of the sensors provide continuous feedback, whereas in another embodiment one or more of the sensors provide feedback at predefined time periods.

### Sparging

As noted above, oxygen is crucial for the cellular processes of respiration and cell division. CO₂ is a waste byproduct of these processes and levels of both of these gases impact the media pH and product quality. Aerobic cells in culture are in a liquid, and therefore oxygen that is available to the cells is dissolved in the media.

A sufficiently high level of dissolved Oxygen (DO) is important to maintain cell growth. However, controlling the upper level of oxygen in a cell culture process is also important because reactive oxygen species can chemically degrade a protein of interest. In some embodiments, a bioreactor as described herein is configured to maintain an operating range of DO between about 30% and about 40%.

Moreover, excessively high levels of CO₂ can be detrimental to cell growth, particularly levels exceeding 20%. In some embodiments, a bioreactor as described herein is configured to maintain a dissolved CO₂ level between about 5% and about 10%.

Sparging involves introduction of air and, more importantly, oxygen, to the cell-culture media. Once the oxygen is dissolved, it is used rapidly by the cells and therefore must be fed continuously via specially designed spargers.

Low oxygen solubility of the culture medium and rapid uptake by cells means that dissolved oxygen (DO) concentration can limit cell growth. Therefore, the manner in which sparging is conducted is critical.

Low oxygen solubility is impacted by interfacial as well as residence time. Smaller bubbles have a larger surface area to volume ratio than larger bubbles, and higher surface area allows oxygen to dissolve more readily and quickly into the culture media. The transfer of oxygen from bubbles to the media also requires adequate time for oxygen to dissolve and transfer and small bubbles rise slowly and therefore have longer residence time.

According to some embodiments, for example as illustrated in FIG. 1A, a bioreactor 100 is provided, comprising:
a vessel 104 having an open top and a vessel wall 105;
a reactor headplate 106 closing the open top of the vessel to the atmosphere;
at least one magnetic stirrer shaft 125a, 125b in the vessel, magnetically coupled to at least one motor 120a, 120b mounted on the headplate 106 outside the vessel; the magnetic stirrer shaft comprising a plurality of impellers 130a, 130b, at least one impeller of said plurality of impellers having an adjustable vertical position on the stirrer shaft;
a sparger 135, comprising:
   a tube assembly 135a inside the vessel, having an outlet;
   a source of sparging gas outside the vessel;
   a conduit 135b, sealed to the atmosphere, opening to the source of sparging gas and to the tube assembly; and
   a sparger driver motor 135c outside the vessel configured to control gas insertion into the tube assembly.

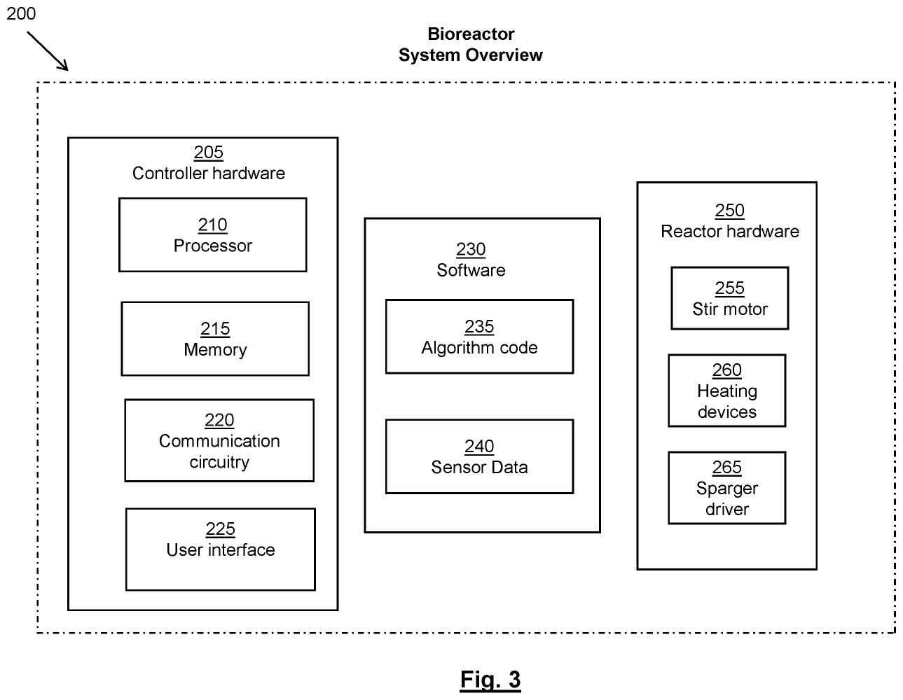

FIG. 3 is a block diagram of the bioreactor system overview, depicting controller hardware 205 and software 230, and reactor hardware 250, according to an embodiment.

Specifically, controller hardware 205 includes one or more processors 210, short-term and long-term memory 215, communication circuity 220 operative to provide wireless communication between the controller and reactor hardware 250 and user interface 225 operative to enable users to input controller operation parameters like a mouse and a keyboard, for example, and to receive sensor outputs through a display screen, for example. Software 230 includes algorithms employed to run the controller interfacing with reactor hardware and sensor data 240. Reactor hardware includes variable-speed, stir motor 255, heating elements and Peltier thermocouple thermocouples 260, and sparger driver 265 implemented as a linear motor, a torque motor, or even a solenoid coil actuator in accordance with the sparger assembly as noted above.

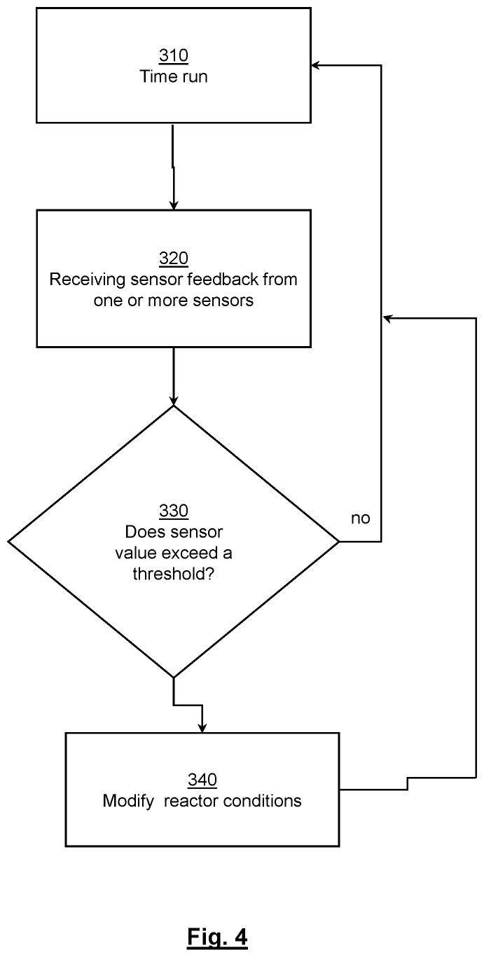

FIG. 4 is a process flow diagram for embodiments employing sensor responsive run-time, parameter optimization. In step 310, run time is clocked so that at pre-defined times sensor feedback is obtain in step 320. In step 330, the sensor feedback is evaluated against a pre-defined threshold value. If the threshold value is exceeded, correct action is taken at step 340 in the form of stirring modification, sparging modification, heating modification or a combination of all of them or any two of them simultaneously or in predefined time sequences. If the threshold characterizing the need for corrective action has not been achieved, then no corrective action is implemented and the controller waits until the next sensor feedback is evaluated. In a certain embodiment, sensor feedback is provided every five minutes, whereas in another embodiment it is provided every minute, while in another embodiment the sensor feedback is obtained every 15 minutes. It should be noted that the time interval at which each specific sensor obtains a measurement is a configurable feature, in a certain embodiment.

Bioreactors as described herein can be scaled as appropriate to achieve a desired size and volume of output, including, without limitation, a small benchtop system or a system adapted to commercial scale. Furthermore, bioreactors as described herein can be operated in any suitable operation mode, including batch operation, continuous operation, or fed-batch operation.

In addition to features specifically depicted in the illustrated embodiments, a bioreactor system in accordance with the presently disclosed subject matter can include, according to some embodiments, other features to optimize functionality, such as appropriate aeration inlets, seal assemblies, bearings, plates, ports, gas flow meters for measuring aeration, electronic or manual flow controllers, tachometers to measure RPM of impellers, or other instruments or devices suitable for maintaining, controlling and assessing the environment. In operation, in an embodiment, RPM can be controlled by varying power to the impeller shaft.

Bioreactors of the presently disclosed subject matter can be used, according to some embodiments, in a variety of cell culture applications and broader bioprocesses. Processes involving the expansion of cells in a bioreactor of the presently disclosed subject matter can include, without limitation, TILs processing, CAR-T/TCR cell processing, adhesion MSCs, adhesion and gene delivery, exosome/secretome, virus processing, or any other type of cells or tissue where such optimized cell expansion is desirable.

In an embodiment, provided herein is a method of culturing cells in a bioreactor as described herein, comprising introducing cells and medium into the bioreactor, introducing oxygen into the bioreactor, and operating the bioreactor as described in accordance with embodiments herein. In an embodiment, culturing comprises suspension cell culturing. In an embodiment, culturing comprises adherent cells culturing. In an embodiment, culturing comprises both suspension cell culturing and adherent cell culturing.

It should be appreciated that embodiments formed from combinations of features set forth in separate embodiments are also within the scope of the presently disclosed subject matter.

While certain features of the presently disclosed subject matter have been illustrated and described herein, modifications, substitutions, and equivalents are included within the scope of the presently disclosed subject matter, mutatis mutandis.

1. A bioreactor comprising:
a vessel defining a cavity therewithin; and
a stirring device comprising at least two stirring elements; the stirring device being configured to operate each of the stirring elements independently of the other to stir media accommodated within the cavity, said stirring elements being configured to rotate about a common stirring axis.

2. The bioreactor according to claim 1, wherein each stirring element comprises a cylindrical stirring shaft attached at its distal end to an impeller located within the cavity, and at its proximal end to a motor, located exterior to the vessel; each motor is configured to independently rotate and/or translate its associated stirring shaft around and/or along the stirring axis, and accordingly its associated stirring impeller.

3. The bioreactor according to claim 2, wherein at least one stirring shaft comprises a hollow cylindrical shape, configured to at least partially accommodate, along its longitudinal axis, at least one other stirring shaft, in a telescopic cylinder configuration, configured to enable the rotation and translation of the at least one other stirring shaft therewithin, and such that their associated impellers reside one next to the other, along the stirring axis.

4. The bioreactor according to claim 3, wherein the bioreactor is configured to be accommodated on a table construction; the table construction comprises at least one motor
shaft per each motor, such that each motor is configured to be applied on and move along its at least one associated motor shaft, and optionally at least one mutual shaft.

5. The bioreactor according to claim 3, further comprising a sleeve tube configured to cover and seal a section of the stirring shafts, the section accommodated within the vessel, such that the stirring shafts are isolated from the vessel's accommodated media, wherein:
the sleeve tube is configured to enable the rotation and translation of the stirring shafts therewithin; and
the impellers are configured to be threaded around the exterior of the sleeve tube; the impellers attachment with the distal end of their associated stirring shafts is via magnetic communication, through the sleeve tube, such that the impellers are enabled to rotate and translate together with their associated stirring shafts.

6. The bioreactor according to claim 5, wherein the sleeve tube comprises a ring bracket at its exterior distal end, configured to prevent the impellers release from the sleeve tube.

7. (canceled)

8. (canceled)

9. (canceled)

10. The bioreactor according to claim 2, wherein the stirring shafts are configured to pass through the headplate, such that media accommodated in the vessel remains sealed therewithin.

11. The bioreactor according to claim 2, wherein at least one stirring shaft is attached to its associated impeller via a magnetic coupling.

12. The bioreactor according to claim 2, wherein at least one stirring shaft is attached to its associated motor via a magnetic coupling and/or a belt coupling.

13. The bioreactor according to claim 1, wherein the stirring device is configured to operate the stirring elements to move along the stirring axis independently of one another or to rotate at different rotational velocities than each other.

14. The bioreactor according to claim 1, wherein the stirring device is configured to operate the stirring elements to move along the stirring axis independently of one another and to rotate at different rotational velocities than each other.

15. The bioreactor according to claim 1, further comprising a sparger.

16. The bioreactor according to claim 1, further comprising sensing elements attached to the vessel's interior surface and their associated readers attached to the vessels exterior surface.

17. The bioreactor according to claim 1, the vessel comprising plurality of ports configured to enable at least one or more selected from the group including: harvesting, washing, sensing, sampling, media refreshing, gas venting, and seeding.

18. The bioreactor according to claim 1, further comprising at least one controller configured to enable automatic maintenance of an accommodated media.

19. (canceled)

20. (canceled)

21. A stirring device, comprising at least two stirring elements; the stirring device being configured to operate each of the stirring elements independently of the other to stir fluid accommodated within a vessel, all the stirring elements being configured to rotate around a common stirring axis.

22. The device according to claim 21, wherein each stirring element comprises a cylindrical stirring shaft attached at its distal end to an impeller, and at its proximal end to a motor; each motor is configured to independently rotate and/or translate its associated stirring shaft around and/or along the stirring axis, and accordingly its associated impeller.

23. The device according to claim 22, wherein at least one stirring shaft is attached to its associated impeller via a magnetic coupling.

24. The device according to claim 22, wherein at least one stirring shaft is attached to its associated motor via a magnetic coupling and/or a belt coupling.

25. The device according to claim 22, wherein at least one stirring shaft comprises a hollow cylindrical shape, configured to at least partially accommodate along its longitudinal axis at least one other stirring shaft, in a telescopic cylinder configuration, configured to enable the rotation and translation of the at least one other stirring shaft therewithin, and such that their associated impellers reside one next to the other, along the stirring axis.

26. The device according to claim 25, wherein the device is configured to be accommodated on a table construction; the table construction comprises at least one motor shaft per each motor, such that each motor is configured to be applied on and move along its at least one associated motor shaft, and optionally at least one mutual shaft.

27. The device according to claim 25, further comprising, a sleeve tube configured to cover and seal a section of the stirring shafts, wherein:
the sleeve tube is configured to enable the rotation and translation of the stirring shafts therewithin; and
the impellers are configured to be threaded around the exterior of the sleeve tube; the impellers attachment with the distal end of their associated stirring shafts is via magnetic
communication, through the sleeve tube, such that the impellers are enabled to rotate and translate together with their associated stirring shafts.

28. The device according to claim 26, wherein the sleeve tube comprises a ring bracket at its exterior end, configured to prevent the impellers release from the sleeve tube.

29. The device according to claim 21, configured to operate the stirring elements to move along the stirring axis independently of one another or to rotate at different rotational velocities than each other.

30. The device according to claim 21, configured to operate the stirring elements to move along the stirring axis independently of one another and to rotate at different rotational velocities than each other.

31. (canceled)

32. (canceled)
